(19) United States
(12) Patent Application Publication
(10) Pub. No.: US 2016/0263341 A1
(43) Pub. Date: Sep. 15, 2016

(54) GAS GENERATOR

(71) Applicant: Hsin-Yung Lin, Shanghai (CN)

(72) Inventor: Hsin-Yung Lin, Shanghai (CN)

(21) Appl. No.: 15/064,558

(22) Filed: Mar. 8, 2016

(30) Foreign Application Priority Data
Mar. 9, 2015 (TW) 104203477
Mar. 9, 2015 (TW) 104203478

(51) Int. Cl. A61M 16/10; C25B 15/02; A61M 16/16; C25B 1/04

(52) U.S. Cl. CPC A61M 16/101; C25B 1/04; C25B 15/02; A61M 16/16

(57) ABSTRACT

A gas generator for health is provided. The gas generator for health includes an electrolysis device, a gas mixing chamber and a gas output device. The electrolysis device is for electrolyzing water to produce a combination gas including hydrogen and oxygen. The gas mixing chamber includes a shell and a cover. The shell has an anti-explosion hole. The cover is separatably disposed on the anti-explosion hole, and the diameter of the anti-explosion hole is larger than 0.5 cm. The gas mixing chamber is connected to the electrolysis device for receiving the combination gas. A vaporized gas is generated by the gas mixing chamber and mixed with the combination gas to produce a healthy gas. The gas output device is connected to the gas mixing chamber for outputting the healthy gas to a user to breath.

### FIELD OF THE INVENTION

The present invention provides a gas generator, especially a gas generator with anti-backfire function.

### DESCRIPTION OF THE PRIOR ART

From ancient times till now, human beings have always considered preserving life as a high priority. Many developments in medical technology are used for treating diseases and increasing life expectancy. In the past, most medical treatment was passive. In other words, diseases are treated only when people fall ill, by performing surgical operation, medication, chemotherapy, radiation treatment and so on. But recently, many medical experts are focusing on disease prevention, such as studying on the effects of health food, and screening for genetic disorders to actively reduce the risk of illness. Furthermore, to increase life expectancy, many anti-aging technologies have been developed, including skin care products and antioxidant food/medicine.

Studies have found that there is an instable oxygen species (O+), also known as free radicals, in the human body. The free radicals are usually generated due to diseases, diet, environment and one's lifestyle, and the free radicals in the human body can be excreted in the form of water by reacting with the inhaled hydrogen. The amount of free radicals in the human body can be reduced, thereby restoring the body condition from an acidic state to an alkaline state, achieving an anti-oxidation, anti-aging and beauty health effects, and even eliminating chronic diseases. Furthermore, there are clinical experiments showing that for patients who need to inhale a high concentration of oxygen for a long time, the lung damage from the high concentration of oxygen can be ameliorated by inhaling hydrogen.

However, the backfire generated by the hydrogen and oxygen combusting towards the direction of the location which generates the hydrogen and oxygen must be avoided when the hydrogen and oxygen device is in use. Namely, the gas generator will explode if the backfire gets into the whistler.

Besides, the gas generator not only comprises the electrolytic cell but the devices needing power like controller and the display, and the electrolytic cell needs larger current and voltage to improve the electrolytic ability, and the other devices generally need the smaller current and voltage than electrolytic cell.

### SUMMARY OF THE INVENTION

According to one aspect of the invention, a gas generator can produce a healthy gas comprising a gas mixture of hydrogen and oxygen and a vaporized gas for being inhaled by a user.

The present invention provides a gas generator for health comprising an electrolysis device, a gas mixing chamber and a gas output device. The electrolysis device is for electrolyzing water to produce a combination gas including hydrogen and oxygen. The gas mixing chamber includes a shell and a cover. The shell has an anti-explosion hole. The cover is separatably disposed on the anti-explosion hole, and the diameter of the anti-explosion hole is larger than 0.5 cm. The gas mixing chamber is connected to the electrolysis device for receiving the combination gas. A vaporized gas is generated by the gas mixing chamber and mixed with the combination gas to produce a healthy gas. The gas output device is connected to the gas mixing chamber for outputting the healthy gas to a user to breath.

The gas generator in an embodiment further comprises a backfire device. The backfire device is disposed between the electrolysis device and the gas mixing chamber. The backfire device comprising an input channel and an output channel is connected with the electrolysis device and the gas mixing chamber by the input channel and the output channel separately.

The gas generator in an embodiment comprises a flow controller connected with the electrolysis device for controlling the flow of the combination gas including hydrogen and oxygen, wherein the flow of the combination gas including hydrogen and oxygen is between 0.01 L/min and 12 L/min.

The flow controller of the gas generator in an embodiment further comprises a flow monitor connected with the electrolysis device, wherein the power of the electrolytic cell is cut by the flow monitor to lower the concentration of oxygen in the combination gas including hydrogen and oxygen when the flow monitor monitors the flow of the combination gas including hydrogen and oxygen of the electrolytic cell is abnormal.

The gas generator in the embodiment further comprises a pressure monitor and a deflation valve, wherein the pressure monitor is connected to the electrolysis device for detecting if the pressure of the electrolysis device is more than a danger value; namely, if the pressure is more than a danger value, the pressure monitor will selectively open the deflation valve to lower the air pressure in the electrolysis device.

The gas generator in the embodiment further comprises an adding gas unit connected with the electrolysis device to add a gas into the combination gas including hydrogen and oxygen to lower the volume concentration of the hydrogen of the combination gas including hydrogen and oxygen. The gas added into the combination gas including hydrogen and oxygen is air, steam, oxygen, inert gas or the combination of air, steam, oxygen and inert gas.

The gas generator of the present invention is able to generate the combination gas including hydrogen and oxygen which is selectively mixed with the steam, the vaporized drug, the vaporized essential oil to generate a healthy gas for the user to breath. Because the healthy gas comprises oxygen, the user breathes the healthy gas to have the antioxidant effect and the anti-aging effect. Otherwise, the healthy gas comprising the vaporized drug helps users absorb the drug. Moreover, the healthy gas comprising the vaporized essential oil helps the user release the stress and improves the health.

The gas generator of the present invention in an embodiment comprises an electrolysis device, a gas mixing chamber, a gas output device and a backfire device. The electrolysis device is for an electrolyzing water to produce a combination gas including hydrogen and oxygen, and the combination gas including hydrogen and oxygen is outputted form an output pipe of the electrolysis device. The gas mixing chamber comprises a shell and a cover. The shell has an anti-explosion hole. The cover is separately disposed on the anti-explosion hole, the gas mixing chamber is connected to the electrolysis device for receiving the combination gas. The vaporized gas is generated by the gas mixing chamber and mixed with the combination gas to produce a healthy gas. The diameter of the anti-explosion hole is larger than 0.5 cm. The gas output device is for receiving the healthy gas and outputting the healthy gas. The backfire device is disposed between the output pipe of the electrolysis device and the gas output device.

The gas generator of the present invention avoids the backfire condition made by the fire moving in the direction of the electrolysis device by the backfire device when oxygen is combusting. To advance the ability of the backfire and lower the occurrence of the air blast, the energy generated by the combustion of oxygen is output from the anti-explosion hole of the cover.

The gas generator of the present invention in an embodiment comprises an electrolysis device, a gas mixing chamber, a gas output device and a power supply. The electrolysis device capable of accommodating the electrolyze water is for electrolyzing water to generate a combination gas including hydrogen and oxygen. The gas mixing chamber is connected to the electrolysis device for receiving the combination gas. A vaporized gas is generated by the gas mixing chamber and mixed with the combination gas to produce a healthy gas. The gas output device is connected to the gas mixing chamber for outputting the healthy gas to a user to breath. The power supply can respectively provide a high electrical current to the electrolysis device and a low electrical current to the gas mixing chamber. The power supply is able to provide the high electrical current and the low electrical current to electrolysis device and the gas mixing chamber separately.

According to the gas generator of the present invention in another embodiment, the power supply comprises a high electrical current output device and a low electrical current output device outputting a high electrical current and a low electrical current, and the power supply further comprises an adjusting device for adjusting the output voltage and output current of the high electrical current output device. The high electrical current output device comprises a first output terminal and a second output terminal, and the power supply further comprises a recorder which is able to record the using duration of the high electrical current output device and the times the high electrical current output device starts. When the using duration and the times of the high electrical current output device to be started are bigger than a predetermined value, the power supply is able to change the polarity of the first output terminal and the second output terminal and reset the recorder.

The gas generator of the present invention in an embodiment further comprises a backfire device. The backfire device is connected between the electrolysis device and the gas mixing chamber, the gas mixing chamber and the gas output device or on the gas output device.

Otherwise, the gas generator of the present invention in another embodiment, the gas mixing chamber comprises a shell and a cover. The shell comprises an explosion-proof vent. The cover is disposed on the explosion-proof vent separately, wherein the diameter of the explosion-proof vent is more than 0.5 cm. The energy generated by the PAHs combusting is able to be output from the explosion-proof vent with cover to improve the anti-backfire function to reduce the damage of the air blast.

The gas generator of the present invention generates the combination gas including hydrogen and oxygen, which is able to mix with vaporized drug, steam or vaporized oil to form the healthy gas for the user to breath. Because there is the healthy gas with predetermined concentration of hydrogen, the user can get the anti-age and antioxidant effects after breathing in. Moreover, the healthy gas is able to comprise vaporized drug to improve the absorption of the drug, and the healthy gas comprises the vaporized oil for releasing the stress and improving the health.

The advantages and spirits of the invention may be understood by the following recitations together with the appended drawings.

### BRIEF DESCRIPTION OF THE DRAWINGS

Some of the embodiments will be described in detail, with reference to the following figures, wherein like designations denote like members, wherein:

FIG. 1 shows the function block diagram of the gas generator of the present invention in an embodiment.

FIG. 2 shows the diagram of the electrolysis device, the flow controller and the adding gas unit of the gas generator of the present invention in an embodiment.

FIG. 3A shows the diagram of the gas mixing chamber disposed on the cover and the gas output device of the gas generator of the present invention in an embodiment.

FIG. 3B shows the gas generator of the present invention without the gas mixing chamber disposed on the cover and the gas output device in an embodiment.

FIG. 4 and FIG. 5 show the gas generator of the present invention in the different angels in an embodiment.

FIG. 6 shows the function block diagram of the gas generator of the present invention in an embodiment.

FIG. 7 shows the diagram of the electrolysis device and the flow controller of the gas generator of the present invention in an embodiment.

FIG. 8 and FIG. 9 show the gas generation of the present invention in the different angels in an embodiment.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

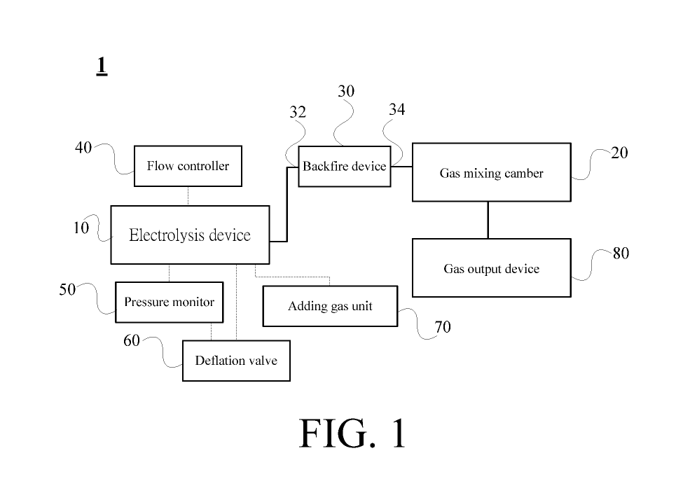

FIG. 1 shows the function block diagram of the gas generator of the present invention in an embodiment. The gas generator 1 comprises an electrolysis device 10, a gas mixing chamber 20 and a gas output device 80. The electrolysis device 10 is for electrolyzing water W to produce a combination gas including hydrogen and oxygen G. The main component of the electrolyzing water W is the water with a little bit of the electrolytic like the sodium hydroxide, the calcium carbonate and the sodium chloride. The gas mixing chamber 20 comprises a shell 22 and a cover 24. The shell 22 comprises an anti-explosion hole 220. The cover 24 is separatably disposed on the anti-explosion hole 220, and the diameter of the anti-explosion hole 220 is larger than 0.5 cm. The gas mixing chamber 20 is connected to the electrolysis device 10 for receiving the combination gas including hydrogen and oxygen G. The gas mixing chamber 20 generates the vaporized gas to mix with the combination gas including hydrogen and oxygen G to generate a healthy gas. The gas output device 80 is connected to the gas mixing chamber 20 for outputting the healthy gas to a user to breath.

The electrolysis device 10 comprises an electrolytic cell 16 comprising an electrode 14A and an electrode 14B; the electrodes 14A and 14B are a cathode electrode and an anode electrode separately and connected with a power source which is not shown in the figures to provide the electrolyzing water power. In some embodiments, the polarity of the electrodes 14A and 14B are fixable; for example, the electrode 14A is a cathode electrode and the electrode 14B is an anode electrode. In other embodiments, the polarity of the electrodes 14A and 14B are changeable; for example, the electrodes 14A and 14B are a cathode electrode and an anode electrode separately at a certain time, and the electrodes 14A and 14B become an anode electrode and a cathode electrode separately after a certain predetermined time; and they can be changeable accordingly.

The electrolyzing water W of the electrolytic cell 16 is electrolyzed when the electrodes 14A and 14B are energized, and the cathode electrode generates the hydrogen and the anode electrode generates oxygen released on the top of the electrolytic cell 16 to generate a combination gas including hydrogen and oxygen G. Besides, the electrolytic cell 16 is able to further comprise an input pipe 12 for adding the electrolyzing water W into the electrolytic cell 16. The combination gas including hydrogen and oxygen G is output by the output pipe 13 of the electrolytic cell 16. In an embodiment, the hydrogen generated by the cathode electrode and oxygen generated by the anode electrode are output to the electrolytic cell 16 through a pipe to mix for generating the combination gas including hydrogen and oxygen. The ratio of the hydrogen and oxygen after the electrolyzing water W which is electrolyzed is 2:1, wherein the ratio of the hydrogen is able to be higher than 66%. In an embodiment, to avoid the hydrogen explosion, an adding gas unit 70 is added into the combination gas including hydrogen and oxygen G to lower the concentration of the hydrogen of the combination gas including hydrogen and oxygen G; for example, the volume concentration of the hydrogen is between 2% and 60% or 2% and 4%. The adding gas is able to be air, steam, oxygen, inert gas or the combination of air, steam, oxygen and inert gas.

The flow of the combination gas including hydrogen and oxygen is lowered to get the effect of the explosion. In an embodiment, the flow controller 40 is disposed on the output pipe 13 for controlling the output flow of the combination gas including hydrogen and oxygen G to lower the concentration of the hydrogen when the combination gas including hydrogen and oxygen G gets into the next device and mixes with the gas in the next device. In an embodiment, the flow controller 40 is able to comprise a flow monitor. Because the oxygen and the hydrogen generated by the electrolytic cell 16 advancing immediately when the power is unstable, the output pipe 13 and the electrolytic cell 16 are able to be disposed by the flow monitor. When the flow of the combination gas including hydrogen and oxygen G generated by the electrolytic cell is more than a dangerous value (the dangerous value is more than 12 L/min or 12000 c.c/min), the flow controller 40 cuts off the power of the electrolytic cell 16 to avoid the concentration of the hydrogen too high to get the control of the output flow of the combination gas including hydrogen and oxygen G; that is to say, the flow controller 40 is able to selectively cut off the power of the electrolytic cell 16. However, the healthy effect decreases if the concentration of the hydrogen is too low. Therefore, the dangerous value mentioned above is better not to be lower than 0.01 L/min; the dangerous value is better between 0.01 L/min and 12 L/min. The flow monitor (or the flow controller 40) is able to be disposed on the other location to monitor the flow of the combination gas including hydrogen and oxygen.

The gas generator of the present invention further comprises a pressure monitor 50 and a deflation valve 60 connected to the pressure monitor 50. The pressure monitor 50 and the deflation valve 60 connected to the electrolysis device 10 are shown in FIG.1, wherein the pressure monitor 50 monitors the air pressure of the electrolysis device 10. When the air pressure is more than a dangerous value, 1 Pa, the pressure monitor 50 opens the deflation valve 60 to make the air pressure of the electrolysis device low to a safe value to avoid explosion. That is to say, the pressure monitor 50 is able to selectively open the deflation valve 60. The pressure monitor 50 and the deflation valve 60 is able to be connected to the other location to monitor the air pressure of the combination gas including hydrogen and oxygen G and lower the air pressure to a safe value. The different combinations of the adding gas unit 70, the flow controller 40 (or the flow monitor), the pressure monitor 50 and the deflation valve 60 mentioned above are able to lower the concentration of the hydrogen to avoid the effect of the explosion.

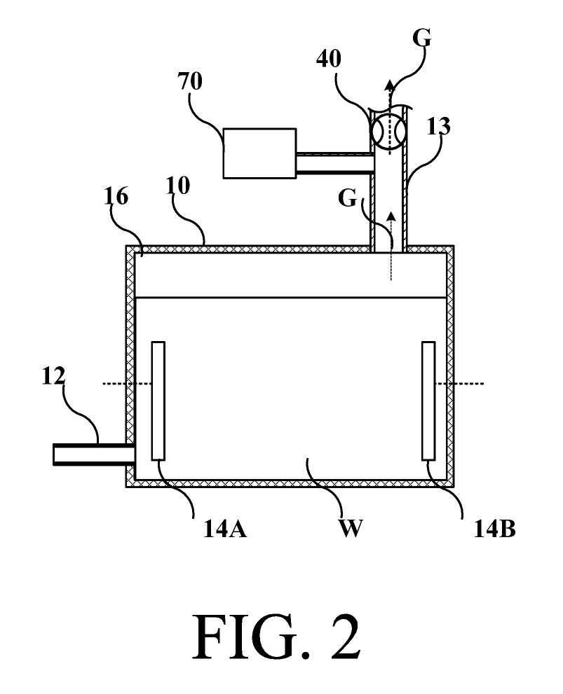

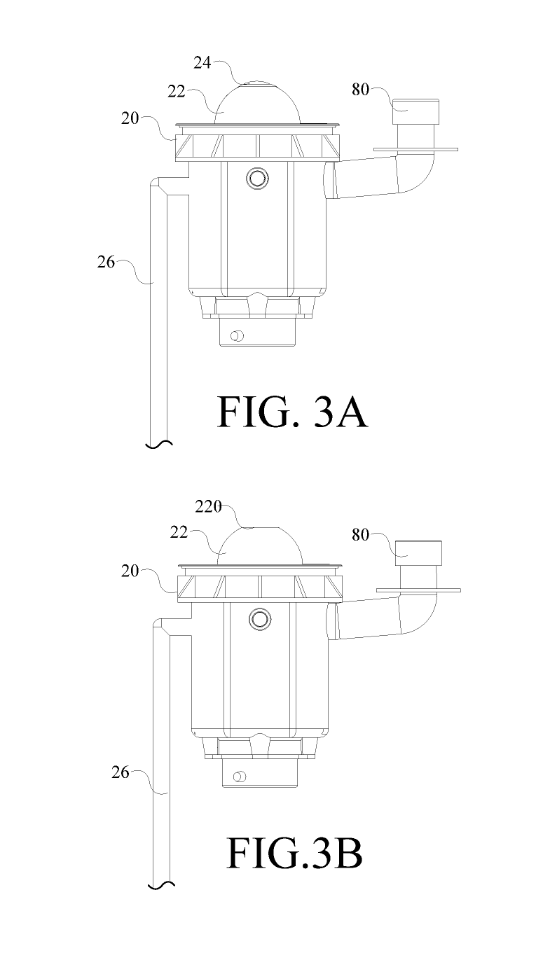

Then, please refer to FIG. 3A and FIG. 3B. FIG. 3A shows the diagram of the gas mixing chamber disposed on the cover and the gas output device of the gas generator of the present invention in an embodiment. FIG. 3B shows the gas generator of the present invention without the gas mixing chamber disposed on the cover and the gas output device in an embodiment. The gas mixing chamber 20 comprises a vaporized/volatile gas mixing chamber (not shown in the figures) and generating a vaporized/volatile gas. In the embodiment, the gas mixing chamber 20 further comprises a pipe 26 connected to the output pipe 13 shown in FIG. 2 for receiving and mixing with the combination gas including hydrogen and oxygen G to generate a healthy gas. Otherwise, the vaporized/volatile gas mixing chamber further comprises an oscillator for vaporizing a first liquid and a second liquid in the vaporized/volatile gas mixing chamber to generate the vaporized/volatile gas. The first liquid is the water which is the base liquid of the oscillate vaporization. The second liquid is able to be the drug, the essential oil, the water and the combination of the drug, the essential oil and the water. That is to say, the vaporized gas is selected from a group comprising steam, vaporized drug, vaporized essential oil and the combination of the steam, the vaporized drug and the vaporized essential oil.

Moreover, the gas mixing chamber 20 comprises a shell 22 and a cover 24. In an embodiment, the shell 22 comprises a semi-circular top cover; in real application, the liquid which is needed to be vaporized to dispose into the vaporized/volatile gas mixing chamber by remove the semi-circular top cover. Moreover, the shell 22 comprises a anti-explosion hole 220 shown in FIG. 3B for the user to drop the liquid which is needed to be vaporized through the taking-up device; the anti-explosion hole also can release the energy when the gas explodes. However, the hole of the anti-explosion hole may release the combination gas including hydrogen and oxygen or the vaporized/volatile gas. Therefore, the cover 24 is able to be separately disposed on the anti-explosion hole 220; in real application, the cover 24 is able to cover, plug and engage onto the anti-explosion hole 220. The healthy gas is output but not released through the direction to the gas output device 80 by sealing anti-explosion hole 220. Moreover, the material of the cover 24 is a rubber like the rubber or the flexible plastic material. The energy is released from the anti-explosion hole 220 of the cover 24 when the hydrogen and oxygen combust. In the embodiment, the diameter of the anti-explosion hole is more than 0.5 cm or 0.8 cm, and the diameter of the anti-explosion hole is between 0.9 cm and 1.5 cm. Moreover, the diameter of the anti-explosion hole is between 0.8 cm and 1.2 cm in a better embodiment. In an embodiment, the material of the cover 24 is a soft rubber, the thickness of the central is between 0.1 mm and 0.2 mm, and the engage portion around the cover 24 engages to the anti-explosion hole 220.

The gas output device 80 is connected to the gas mixing chamber 20 to output the healthy gas for the user to breath.

Besides, in an embodiment, the gas generator further comprises a backfire device 30 for avoiding the condition of the backfire. The backfire device 30 is able to be disposed between the output pipe of the electrolysis device and the gas output device. In an embodiment, the backfire device 30 is connected between the electrolysis device 10 and gas mixing chamber 20. The backfire device 30 comprises an input channel 32 and an output channel 34 shown in FIG. 1. The backfire device 30 is able to be connected to the electrolysis device 10 by the input channel 32, and the backfire device 30 is able to be connected to the pipe of the gas mixing chamber 20 by the output channel 34. In an embodiment, the anti-backfire chamber is disposed between the input channel 32 and the output channel 34 like the metal anti-backfire chamber. When the hydrogen and oxygen combust, the gas generator of the present invention avoids the backfire made by the fire firing through the direction to the electrolysis device by the backfire device 30, and the energy is released from the anti-explosion hole 220 of the cover 24 to improve the degree of the backfire prevention and lower the occurrence of the gas explosion.

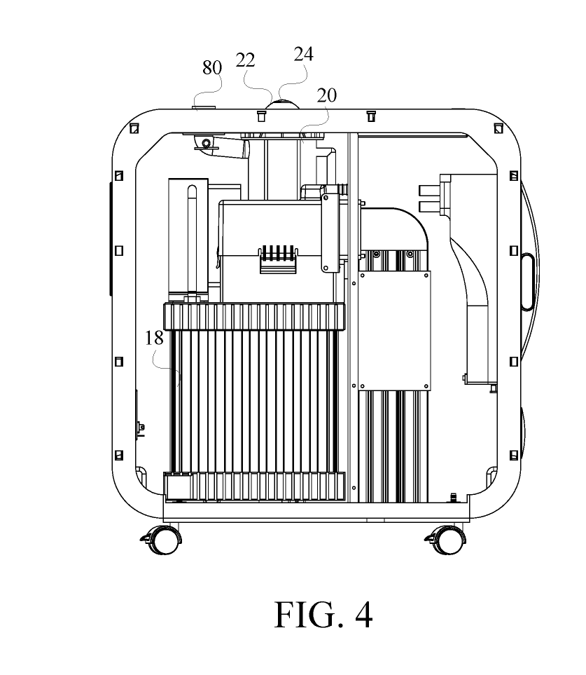

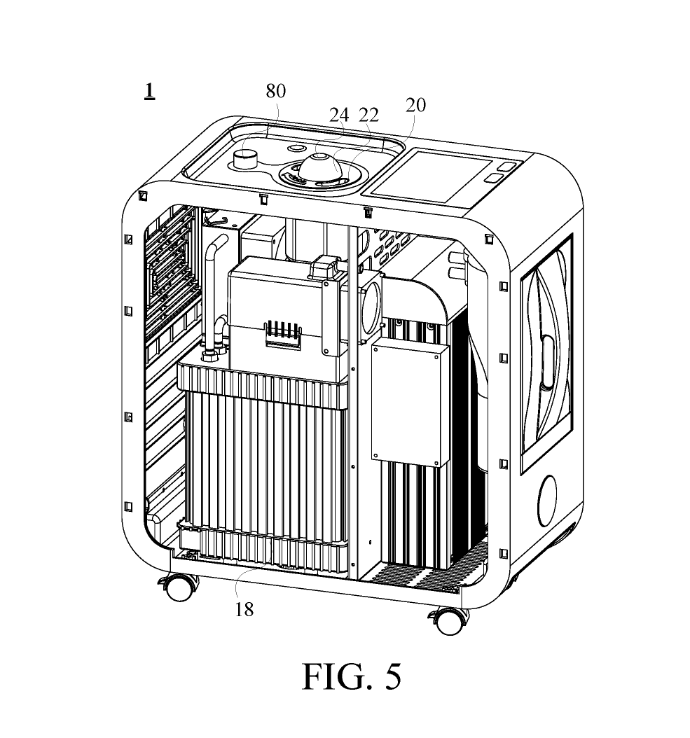

Please refer to FIG. 4 and FIG. 5. FIG. 4 and FIG. 5 show the gas generator of the present invention in the different angels in an embodiment. In an embodiment, the gas generator of the present invention comprises the water tank, the electrolysis device 10 (in the embodiment, the electrolysis device is not shown in the figures because the electrolysis device is disposed in the water tank), the gas mixing chamber 20, the water pump, the condensation filter, the cooling device and the hygroscopicity device. FIG. 4 and FIG. 5 show the relative position of the electrolysis device 10 disposed in water van, the gas mixing chamber 20 and the gas output device 80.

In an embodiment, when the electrolysis device is connected to the outer power source, the output voltage of the power source is between 17 volts and 27 volts, and the output current of the power source is between 30 amperes and 40 amperes. That is to say, the output gas generated by the electrolysis device is 1.5 L/min and 4.0 L/min. The voltages between the electrodes are 1.5 volts and 3 volts when the electrodes are in use, and the voltage is between 12 volts and 24 volts if there are eight pairs of the electrodes. But the voltage, the output current and the output gas are not limited to it in the real application. When the electrolysis device is connected with the outer power source, the output voltage is between 5 volts and 24 volts, and the output current of the power source is between 2 amperes and 150 amperes. That is to say, the power of the electrolysis device is between 10 watts and 3600 watts, and the output gas of the electrolysis device is between 0.01 L/min and 12 L/min.

From above mentioned, the gas generator of the present invention is able to generate the combination gas including hydrogen and oxygen which is selectively mixed with the steam, the vaporized drug, the vaporized essential oil to generate a healthy gas for the user to breath. Because the healthy gas comprises oxygen, the user breathes the healthy gas to have the antioxidant effect and the anti-aging effect. Otherwise, the healthy gas comprising the vaporized drug helps user absorb the drug. Moreover, the healthy gas comprising the vaporized essential oil helps the user release the stress and improves the health.

Moreover, the gas generator of the present invention avoids the backfire condition made by the fire moving in the direction of the electrolysis device by the backfire device when oxygen is combusting. To advance the ability of the backfire and lower the occurrence of the air blast, the energy generated by the combustion of oxygen is output from the anti-explosion hole of the cover.

To summarize the statement mentioned above, the gas vending system for health application of the present invention can use a charging/payment module to charge according to the toll that corresponds to the volume used by the user. The charging system is adapted to accept cash or charge a deposit member card/debit card/credit card conveniently. In addition, through the user management module and because the communication module is connected to the database, the usage state of the user can be completely recorded in order to provide references for the user or the manager of the system. The gas vending system for health application of the present invention further comprises a video/internet module and display device for the user to watch videos or surf the Internet while it is being used, and simultaneously provides a comfortable situation for the user.

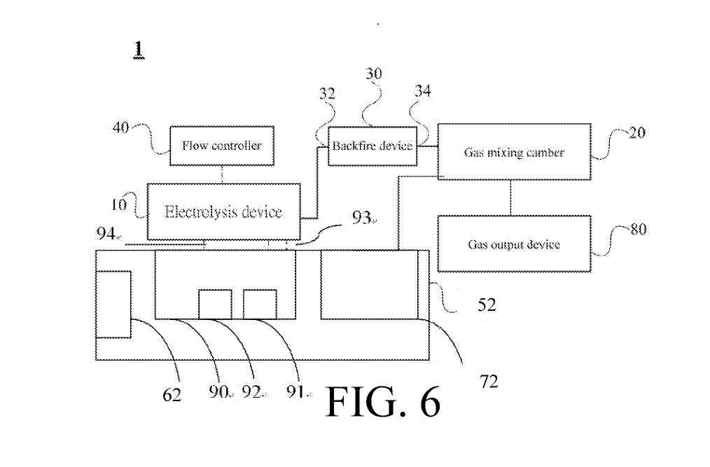

Please refer to FIG. 6; FIG. 6 shows the function block diagram of the gas generator of the present invention in an embodiment. The gas generator 1 comprises an electrolysis device 10, a gas mixing chamber 20, a gas output device 80 and a power supply 52. The electrolysis device 10 is able to accommodate an electrolyze water W for electrolyzing the electrolyze water W to generate a combination gas including hydrogen and oxygen G. The main material of the electrolyze water W is pure water; however, the light electrolytic like sodium hydrate, calcium carbonate and sodium chloride is able to be added according to the different needs. The gas mixing chamber 20 receives the combination gas including hydrogen and oxygen G generated by the electrolysis device 10. The gas mixing chamber 20 generates a vaporized gas mix with the combination gas including hydrogen and oxygen G to form a healthy gas. The gas output device 80 outputs the healthy gas from the gas mixing chamber 20 for the user to breath.

The power supply 52 comprises the power input device 62 and the low electrical current output device outputting the first low electrical current to gas mixing chamber 20 or the device in the gas generator like the display unshown in the figure. The power input device 62 is able to receive the alternating current with the frequency is in a range from 50 Hz to 60Hz and the voltage is in a range from 90V to 250V or other input power with different scale. The low electrical current output device 72 is connected to the power input device 62 to generate the first low electrical current. In an embodiment, the low electrical current output device 72 is able to generate the direct output voltage in a range from 12V to 24V and the first low electrical current in a range from 0.1 A to 5 A. In an embodiment, the low electrical current output device 72 outputs the direct voltage 24V and the first low electrical current in a range from 0.1 A to 5 A. The electrolysis device 10 needs larger current to improve the electrolysis ability. The power supply 52 of the gas generator comprises the high electrical current output device 90 outputting the first high electrical current to electrolysis device 10, wherein the high electrical current output device 90 is able to generate the direct output voltage in a range from 15V to 30V and the first high electrical current in a range from 35 A to 55 A. In an embodiment, the value of the first high electrical current is in a range from 6 times to 40 times to the value of the first low electrical current, more than 10 times in the general condition, to reduce the needs of the electrolytic of the electrolysis device 10.

The power supply 52 further comprises the adjusting device 91 which is able to adjust the output voltage and output current of the high electrical current output device. For example, a knob is disposed on the surface of the gas generator 1 to control the adjusting device 91 to adjust the output voltage of the high electrical current output device 90 in a range from 17V to 26V, and the value of a high electrical current is fixed in a range from 40 A to 50 A. In another embodiment, the gas generator 1 comprises a touch screen and a touch control, the user can touch the touch control to control the adjusting device 91 to adjust the output voltage of the high electrical current output device 90 in a range from 17V to 26V or the high electrical current in a range from 40 A to 50 A. In an embodiment, when the output voltage of the high electrical current output device 90 is in a range from 17V to 27V and the output current of the power source is in a range from 30 A to 40 A, the electrolysis device 10 generates the output gas under the speed in a range from 1.5 L/min to 4.0 L/min.

The high electrical current output device comprises a first output terminal 93 and a second output terminal 94; the power supply further comprises a recorder 92 recording the using duration and the times of the high electrical current output device 90. When the using duration and the times of the high electrical current output device 90 is larger than a predetermined value, the power supply is able to change the polarity of the first output terminal 93 and the second output terminal 94. For example, when the gas generator is started, the first output terminal 93 is the anode and the second output terminal 94 is the cathode. When the gas generator is turned off and started again, the first output terminal 93 turned into the cathode and the second output terminal 94 turned into the anode. In another embodiment, when the times of the gas generator to be started recorded by the recorder 92 is larger than a predetermined time (odd or even, like 3 times), the first output terminal 93 turns into the cathode and the second output terminal 94 turns into the anode. Afterwards, the recorder 92 cleans the records and recounts the times. But when the times of the gas generator to be started recorded by recorder 92 is larger than a predetermined value like 3 again, the first output terminal 93 turns back to the anode and the second output terminal 94 turns back to the cathode again. In another embodiment, when the using duration of the gas generator recorded by the recorder 92 is larger than a predetermined time like 60 minutes, the first output terminal 93 turns into the cathode and the second output terminal 94 turns into the anode. Afterwards, the recorder 92 cleans the records and recounts the using duration. But if the using duration is larger than a predetermined time like 60 minutes again, the first output terminal 93 turns back to the anode and the second output terminal 94 turns back to the cathode. Namely, the recorder is able to comprise the buffer or the other memory devices.

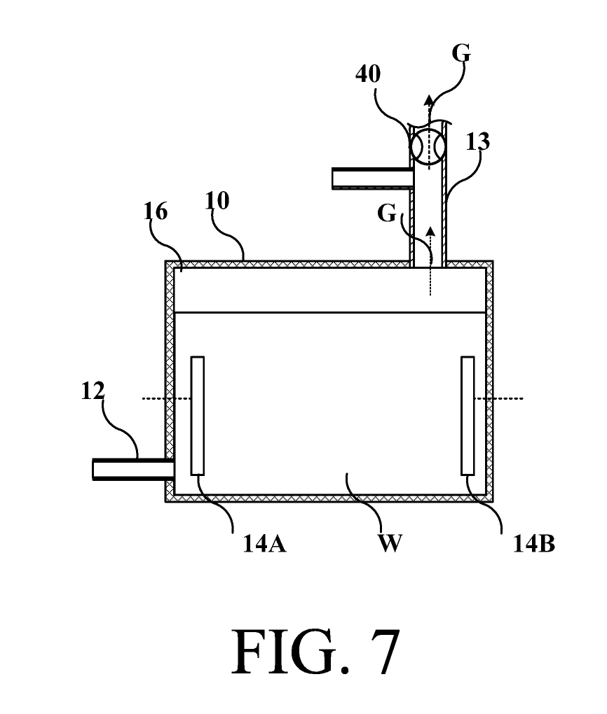

Please refer to FIG. 7; FIG. 7 shows the diagram of the electrolysis device and the flow controller of the gas generator of the present invention in an embodiment. The electrolysis device 10 comprises an electrolytic cell 16, electrolytic cell 16 further comprises an electrode 14A and an electrode 14B, and the electrodes 14A and 14B are coupled to the first output terminal 93 and the second output terminal 94 of the power supply 52 separately to provide the power electrolyze water needs. According to the above-mentioned polarity of the first output terminal 93 and the second output terminal 94 which is changeable, the polarity of the electrodes 14A and 14B are changeable, too. For example, at a certain time, the electrode 14A is the anode and the electrode 14B is the cathode; when the times of the gas generator to be started is larger than the predetermined times or the predetermined using time, the electrode 14A turns into the cathode and the electrode 14B turns into the anode, and the records of the recorder 92 is reset or cleaned.

The electrolyze water W in the electrolytic cell 16 generates hydrogen at the cathode and oxygen at the anode after the electrodes 14A and 14B is energized. Hydrogen and oxygen is released on the electrolytic cell 16 and form a combination gas including hydrogen and oxygen G. Besides, the electrolytic cell 16 further comprises an input tube 12 for providing the electrolyze water W in the electrolytic cell 16. The combination gas including hydrogen and oxygen G is outputted by the output tube 13 of the electrolytic cell 16. In another embodiment, hydrogen and oxygen generated on the cathode and the anode separately are able to be outputted to the electrolytic cell 16 via a gas tube separately and mixed to form the combination gas including hydrogen and oxygen. The ratio of hydrogen and oxygen is 2:1, that is to say hydrogen is about 66% after the electrolyze water W is electrolyzed.

In another embodiment, a flow controller is disposed in the output tube 13 to control the output flow of the combination gas including hydrogen and oxygen G to get the combination gas including hydrogen and oxygen G into the next device to be mix with the gas, and the concentration of hydrogen is able to be reduced. In an embodiment, the flow controller 40 is able to comprise a flow detector disposed in the output tube 13 or the electrolytic cell 16 because sometimes the power is unstable to suddenly make the amount of the PAHs electrolyzed by the electrolytic cell 16 high. When the flow of the combination gas including hydrogen and oxygen G generated by the electrolytic cell is higher than a predetermined value like 12 L/min or 12000c.c/min, the flow controller 40 cuts off the power of the electrolytic cell 16 to avoid the concentration of hydrogen too high to achieve the flow control of the combination gas including hydrogen and oxygen G; that is to say, the flow controller 40 is able to cut off the power of the electrolytic cell 16 selectively. However, if the concentration of hydrogen is too low, the health impact will not be significant. Therefore, the predetermined value is better not to be lower than 0.01 L/min; the better predetermined value is in a range from 0.01 L/min to 12 L/min generally. The flow detector (or the flow controller 40) is able to be disposed on other location where is able to let the flow detector (or the flow controller 40) detect the flow of the combination gas including hydrogen and oxygen.

Moreover, gas mixing chamber 20 comprises a shell 22 and a cover 24. In an embodiment, the shell 22 comprises a half-circle cover. In the real application, the vaporized gas is put into the gas mixing chamber by removing the half-circle cover. Moreover, the shell 22 comprises an explosion-proof vent 220 shown in FIG. 3B, and the liquid needing to be vaporized is dropped into the explosion-proof vent 220 via the suction device. When air blast occurs, the energy is able to get out through the explosion-proof vent. However, the hole of the explosion-proof vent may let the combination gas including hydrogen and oxygen or the vaporized gas leak. In the present invention, the cover 24 is disposed on the explosion-proof vent 220 separately. In real application, the cover 24 is able to cover, to plug or to engage the explosion-proof vent 220. The healthy gas is output toward the gas output device 80 and is not leaked through the explosion-proof vent 220 by sealing the explosion-proof vent. Moreover, the cover 24 could be made of a rubber material like rubber and flexible plastic material. When PAHs is combusting, the energy generated by the combustion of PAHs gets out from the explosion-proof vent 220 of the cover 24. In the embodiment, the diameter of the explosion-proof vent is larger than 0.5 cm; moreover, in a batter embodiment, the diameter of the explosion-proof vent is in a range from 1 cm to 1.2 cm.

The gas output device 80 is connected to the gas mixing chamber 20 to output the healthy gas for the user to breath.

Besides, in an embodiment, the gas generator further comprises a backfire device 30 for avoiding the backfire. The backfire device 30 is able to be disposed between the electrolysis device 10 and the gas mixing chamber 20, between the gas mixing chamber 20 and the gas output device 80, or on the gas output device 80. In an embodiment, the backfire device 30 is disposed between the electrolysis device 10 and the gas mixing chamber 20, and the backfire device 30 comprises an input channel 32 and an output channel 34 shown in FIG. 6. The backfire device 30 is able to be connected to the electrolysis device 10 and the gas mixing chamber 20 via the input channel 32 and the output channel 34 separately. In an embodiment, the anti-backfire chamber is disposed between the input channel 32 and the output channel 34, and the metal anti-backfire net is able to be disposed in the anti-backfire chamber. When PAHs combusts, the gas generator of the present invention is able to avoid the backfire happened by the fire firing toward the electrolysis device 10 by the backfire device 30, and the energy generated by the combustion of PAHs can get out from the explosion-proof vent 220 of the cover 24 to improve the anti-backfire ability and reduce the air-blast damage.

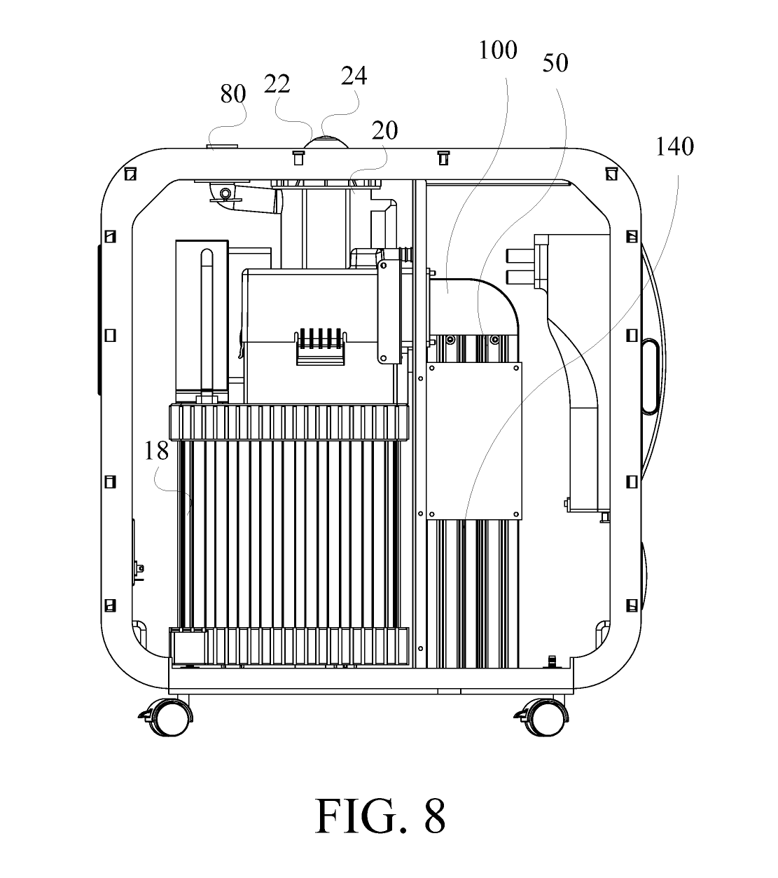

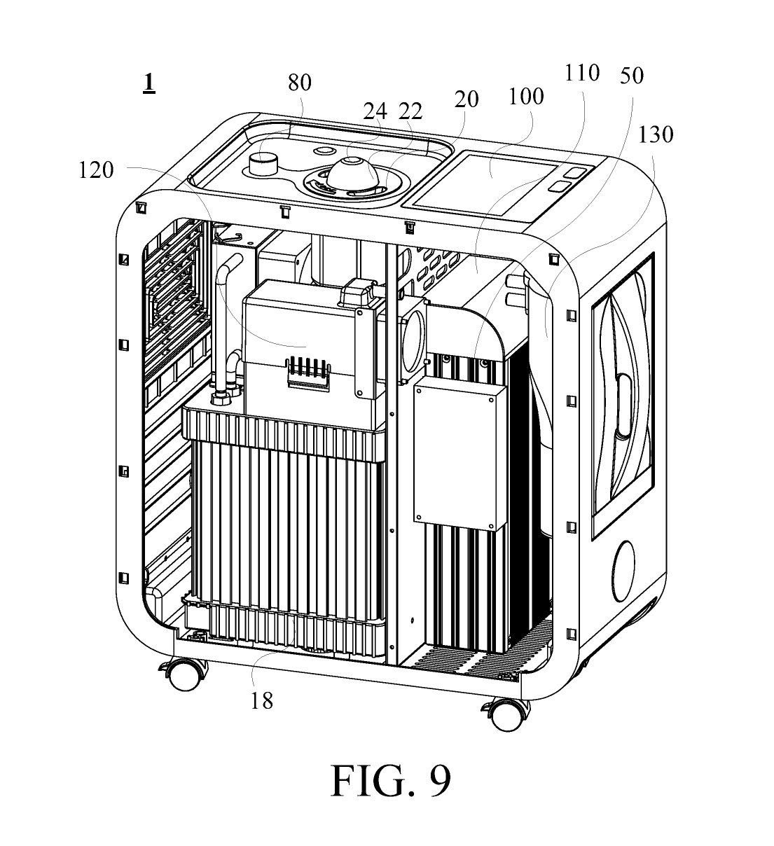

Please refer to FIG.8 and FIG. 9. FIG. 8 and FIG. 9 show the gas generation of the present invention with different angels in an embodiment. FIG. 8 shows the relative position between the electrolysis device 10 disposed in the water tank 18, the gas mixing chamber 20 and the gas output device 80. In an embodiment, the gas generator of the present invention comprises the electrolysis device 10 (in the embodiment, the electrolysis device is disposed in the water tank and unshown in the figure), the gas mixing chamber 20, the power supply 52, the display 110, the filter 120 and the humidified device 130. The power supply 52 comprises the heat sink casing 140 covering all of the elements in the power supply 52 (like the power input device, the low electrical current output device, the high electrical current output device, the adjusting device and the recorder) for improving the ability of dispersing the heat. Otherwise, the power supply 52 further comprises the protective cover 100 for avoiding the water leaking into the power supply 52, and the protective cover is able to be disposed on the heat sink casing.

To summarize the statement mentioned above, the gas generator of the present invention is able to generate the combination gas including hydrogen and oxygen and be mixed with vaporized drug, steam or vaporized oil to form a healthy gas for the user to breath. Because there is the healthy gas with predetermined concentration of hydrogen which is able to help the user to get the anti-age and antioxidant effects after the user breathes in. The healthy gas is able to comprise vaporized drug to improve the absorption of the drug. Moreover, the healthy gas comprises the vaporized oil for releasing the stress and improving the health.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims. Instead, the invention should be determined entirely by reference to the claims that follow.

The embodiments of the invention in which an exclusive property or privilege is claimed are defined as follows:

1. A gas generator, comprising:
an electrolysis device, for an electrolyzing water to generate a combination gas including hydrogen and oxygen;
a gas mixing chamber, comprising a shell and a cover, the shell having an anti-explosion hole, the cover separatably disposed on the anti-explosion hole, the gas mixing chamber connected to the electrolysis device for receiving the combination gas, a vaporized gas generated by the gas mixing chamber and mixed with the combination gas to generate healthy gas, and the diameter of the anti-explosion hole larger than 0.5 cm; and
a gas output device, connected to the gas mixing chamber for outputting the healthy gas to a user to breath.

2. The gas generator of claim 1, wherein the diameter of the anti-explosion hole is between 0.8 cm and 1.2 cm.

3. The gas generator of claim 1, wherein the material of the cover is a rubber.

4. The gas generator of claim 1, wherein the vaporized gas is selected from a group comprising steam, vaporized drug, vaporized essential oil and the combination of the steam, the vaporized drug and the vaporized essential oil.

5. The gas generator of claim 1, wherein the gas generator comprises a backfire device disposed between the electrolysis device and the gas mixing chamber.

6. The gas generator of claim 5, wherein the backfire device comprises an input channel and an output channel, the backfire device is connected to the electrolysis device and the gas mixing chamber by the input channel and the output channel separately.

7. The gas generator of claim 1, wherein the gas generator further comprises a flow controller connected to the electrolysis device for controlling the flow of the combination gas including hydrogen and oxygen, wherein the flow of the combination gas including hydrogen and oxygen is between 0.01 L/min and 12 L/min.

8. The gas generator of claim 1, wherein the flow controller comprises a flow monitor for monitoring the flow of the combination gas including hydrogen and oxygen, and the flow controller is able to selectively cut off the electrolysis device to be disconnected to a power source.

9. The gas generator of claim 1, wherein the gas generator further comprises a pressure monitor connected to a deflation valve, the pressure monitor is connected to the electrolysis device for monitoring if the air pressure of the electrolysis device is more than a danger value, and the pressure monitor can selectively open the deflation valve.

10. The gas generator of claim 1, wherein the gas generator further comprises an adding gas unit connected to the electrolysis device for adding a gas into the combination gas including hydrogen and oxygen to lower the volume concentration of hydrogen of the combination gas including hydrogen and oxygen, wherein the gas added into the combination gas including hydrogen and oxygen is selected from a group comprising air, steam, oxygen, inert gas or the combination of air, steam, oxygen and inert gas.

11. A gas generator, comprising:
an electrolysis device, for an electrolyzing water to generate a combination gas including hydrogen and oxygen, the combination gas including hydrogen and oxygen outputted from an output pipe of the electrolysis device;
a gas mixing chamber, comprising a shell and a cover, the shell having an anti-explosion hole, the cover separatably disposed on the anti-explosion hole, the gas mixing chamber connected to the electrolysis device for receiving the combination gas, a vaporized gas generated by the gas mixing chamber and mixed with the combination gas to generate healthy gas, and the diameter of the anti-explosion hole larger than 0.5 cm;
a gas output device, for receiving the healthy gas and outputting the healthy gas; and
a backfire device, disposed between the output pipe of the electrolysis device and the gas output device.

12. The gas generator of claim 11, wherein the material of the cover is a rubber, and the thickness of the center of the cover is between 0.1 mm and 2 mm.

13. A gas generator, comprising:
an electrolysis device, for accommodating electrolyzed water and electrolyzing the electrolyzed water to generate combination gas including hydrogen and oxygen;
a gas mixing chamber, for receiving the combination gas including hydrogen and oxygen and generating vaporized gas mixed with the combination gas including hydrogen and oxygen to form healthy gas; and
a power supply, comprising a power input device, a low electrical current output device generating a first low electrical current, a high electrical current output device generating a first high electrical current for the electrolysis device, an adjusting device for adjusting an output voltage or the first high electrical current of the high electrical current output device, and a recorder for recording the times of the gas generator to be started and the using duration, wherein the power input device is coupled to the low electrical current output device and the high electrical current output device, and the first low electrical current is provided for the power gas mixing chamber.

14. The gas generator of claim 13, wherein the high electrical current output device comprises a first output terminal and a second output terminal to be controlled to change the polarity by the recorder.

15. The gas generator of claim 14, wherein the recorder changes the polarity of the first output terminal and the second output terminal according to whether the times of the gas generator to be started or the using duration is larger than a predetermined value.

16. The gas generator of claim 13, wherein the adjusting device is coupled to a knob or a touch device of the gas generator, and the adjusting device adjusts the output voltage of the high electrical current output device in a range from 17V to 26V according to the control of the knob or the touch device.

17. The gas generator of claim 13, wherein the power supply further comprises a heat sink case for covering the power input device, the low electrical current output device, the high electrical current output device, the adjusting device and the recorder.

18. The gas generator of claim 17, wherein the power supply further comprises a protective cover disposed on the heat sink case.

19. The gas generator of claim 13, wherein the value of the first high electrical current is in a range from 10 times to 40 times of the value of the low electrical current.

20. The gas generator of claim 13, wherein the first high electrical current is in a range from 40 A to 50 A.
